SEQUENCE LISTING (8 sequences)

SEQ ID NO: 1 - DNA, 21 bp, Artificial Sequence
  Description of Artificial Sequence Synthetic primer
ctgtcccttttcctgcccccg

SEQ ID NO: 2 - DNA, 21 bp, Artificial Sequence
  Description of Artificial Sequence Synthetic primer
agccagacaccgctgccattg

SEQ ID NO: 3 - DNA, 23 bp, Artificial Sequence
  Description of Artificial Sequence Synthetic primer
accttttccttctccwggktcsg

SEQ ID NO: 4 - DNA, 23 bp, Artificial Sequence
  Description of Artificial Sequence Synthetic primer
ccgctctcatatgcytcgacakt

SEQ ID NO: 5 - DNA, 23 bp, Artificial Sequence
  Description of Artificial Sequence Synthetic primer
gtaaaggtctctgacttttggac

SEQ ID NO: 6 - DNA, 24 bp, Artificial Sequence
  Description of Artificial Sequence Synthetic primer
tggaatagaaccttcatcttcacc

SEQ ID NO: 7 - DNA, 24 bp, Artificial Sequence
  Description of Artificial Sequence Synthetic primer
ttggcttcgatattttcagtatct

SEQ ID NO: 8 - DNA, 25 bp, Artificial Sequence
  Description of Artificial Sequence Synthetic primer
cataacaaacatatgcccatttccg

(19) United States
(12) Patent Application Publication
(10) Pub. No.: US 2015/0299775 A1
(43) Pub. Date: Oct. 22, 2015

(54) PCR PRIMERS FOR DETECTION OF VIBRIO PARAHAEMOLYTICUS THERMOSTABLE DIRECT HEMOLYSIN (TDH) AND TDH-RELATED HEMOLYSIN GENES

(71) Applicants: Charles R. Lovell, Batesburg, SC (US); Casandra Karen Gutierrez-West, Eastover, SC (US)

(72) Inventors: Charles R. Lovell, Batesburg, SC (US); Casandra Karen Gutierrez-West, Eastover, SC (US)

(21) Appl. No.: 14/426,600

(22) PCT Filed: Sep. 6, 2012
(86) PCT No.: PCT/US12/53824

§ 371 Date: Jun. 18, 2015

(51) Int. Cl. C12Q 1/68

(52) U.S. Cl. CPC C12Q 1/689

(57) ABSTRACT

Disclosed are PCR primers as may be utilized in detection of the genes encoding the thermostable direct hemolysin (tdh) and the TDH-related hemolysin (trh). The PCR primers can be utilized to detect the presence of pathogenic V. parahaemolyticus in a test sample. Also disclosed are methods for utilizing the PCR primers and kits that include the PCR primers.

### SEQUENCE LISTING

The instant application contains a Sequence Listing which has been submitted electronically in ASCII format and is hereby incorporated by reference in its entirety. Said ASCII copy, created on May 14, 2015, is named USC-337-PCT-US_SL.txt and is 2,198 bytes in size.

### STATEMENT REGARDING FEDERALLY SPONSORED RESEARCH OR DEVELOPMENT

This invention was made with government support under Award No. OCE 0928002 awarded by the National Science Foundation. The government has certain rights in the invention.

### BACKGROUND

Vibrio parahaemolyticus is a gram negative, motile, slightly curved rod belonging to the class Gammaproteobacteria and is the most common bacterium implicated in acute seafood borne gastroenteritis in humans, with an estimated 4500 cases in the United States each year (FDA, 2005). Symptoms of V. parahaemolyticus food poisoning include headache, diarrhea (with or without blood in the stool), nausea, vomiting and abdominal pain (FDA, 2005; Su et al., 2007). In severely immunocompromised individuals this gastroenteritis can lead to septicemia and death. Infection is most often attributed to the mishandling or consumption of raw or undercooked oysters, mussels, fish, crabs and other crustaceans. Occasional V. parahaemolyticus wound infections followed by septicemia have been reported. Three such cases were reported after Hurricane Katrina and two of these were fatal (CDC, 2005).

Strains of V. parahaemolyticus isolated from gastroenteritis patients almost always carry the gene encoding the thermostable direct hemolysin (tdh), the gene encoding the TDH-related hemolysin (trh), or both genes (Shirai et al, 1990, Joseph et al, 1982). Detection of either gene in any strain has long been considered evidence for the potential pathogenicity of that strain (Honda et al, 1992; Raimondi, 2000) and is typically employed in the description of new pathogenic strains. Gene products of tdh and trh are essential to hemolytic activity, as indicated by the Kanagawa phenomenon (1-hemolysis) on Wagatsuma Agar (Ohnishi et al., 2011, Yanagihara et al., 2010). TDH and TRH are tetrameric proteins that form pores of approximately 2 nm in susceptible host cell membranes (Honda et al, 1992; Kishishita, 1993). This causes a non-specific efflux of divalent cations (Raimondi, 2000) or, in the case of human erythrocytes, swelling due to water influx followed by cell lysis (Honda et al, 1992). Further, the purified toxins have been demonstrated to be lethal to laboratory animals when injected intravenously or intraperitoneally (Honda et al., 1976a). Strains of V. parahaemolyticus isolated from environmental materials have rarely been reported to contain either tdh or trh and are generally considered non-pathogenic (Vieira et al, 2011, FDA 2005, Nishibuchi and Kaper, 1995). In contrast to this view Velazquez-Roman et al. (2012) recently reported that 52% of environmentally isolated strains from the Pacific coast of Mexico contained either tdh or trh. Differences in the frequency of detection of the tdh and trh genes could result from inconsistent PCR amplification, leading to inaccurate conclusions regarding the connection between the presence of these genes and the probable pathogenicity of strains that contain them.

What are needed in the art are materials that can provide more accurate detection of pathogenic strains of V. parahaemolyticus. More specifically, what are needed are PCR primers for use in PCR amplification of tdh and trh that can provide improved accuracy in screening methods and can facilitate detection of pathogenic strains of V. parahaemolyticus.

### SUMMARY

According to one embodiment, disclosed are PCR primers for detecting pathogenic strains of V. parahaemolyticus. The PCR primers include SEQ ID NO: 1, SEQ ID NO: 2, SEQ ID NO: 3, and SEQ ID NO: 4.

Also disclosed is a kit for detecting pathogenic strains of V. parahaemolyticus, the kit includes a pair of PCR primers, the pair of PCR primers consisting of SEQ ID NO: 1 and SEQ ID NO: 2 or SEQ ID NO: 3 and SEQ ID NO: 4. In one embodiment, a kit can include both primer pairs, i.e., a first pair of PCR primers consisting of SEQ ID NO: 1 and SEQ ID NO: 2, and a second pair of PCR primers consisting of SEQ ID NO: 3 and SEQ ID NO: 4

Methods for detecting pathogenic strains of V. parahaemolyticus are also disclosed. For instance, a method can include amplifying a tdh sequence and/or a trh sequence contained in a test sample by use of the disclosed PCR primers.

### BRIEF DESCRIPTION OF THE FIGURE

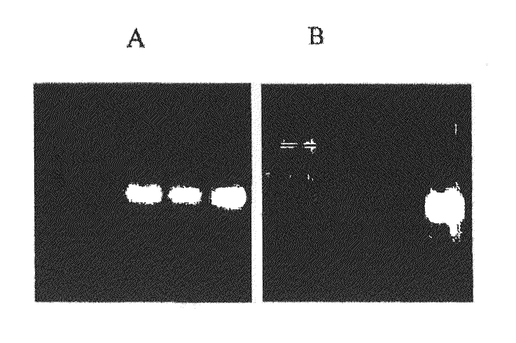

The present application may be better understood with reference to FIG. 1, which is an agarose gel image of tdh amplicons of environmental strains of V. parahaemolyticus (lanes 1 and 2) and the positive control V. parahaemolyticus strain ATCC33846 (lane 3) amplified using SEQ ID NO: 1/SEQ ID NO: 2 (FIG. 1A) and tdh amplicons of environmental strains of V. parahaemolyticus (lanes 1 and 2) and ATCC 33846 (lane 3) amplified using comparative PCR primers SEQ ID NO: 5/SEQ ID NO: 6 (FIG. 1B).

### BRIEF DESCRIPTION OF THE SEQUENCE LISTING

SEQ ID NO: 1 is a PCR primer as may be used to amplify tdh as disclosed herein.

SEQ ID NO: 2 is another PCR primer as may be used to amplify tdh as disclosed herein.

SEQ ID NO: 3 is another PCR primer as may be used to amplify trh as disclosed herein.

SEQ ID NO: 4 is another PCR primer as may be used to amplify trh as disclosed herein.

SEQ ID NO: 5 is a previously known PCR primer used for comparison to the disclosed PCR primers.

SEQ ID NO: 6 is a previously known PCR primer used for comparison to the disclosed PCR primers.

SEQ ID NO: 7 is a previously known PCR primer used for comparison to the disclosed PCR primers.

SEQ ID NO: 8 is a previously known PCR primer used for comparison to the disclosed PCR primers.

### DETAILED DESCRIPTION

Reference will now be made in detail to various embodiments of the disclosed subject matter, one or more examples of which are set forth below. Each embodiment is provided by way of explanation of the subject matter, not limitation of the subject matter. In fact, it will be apparent to those skilled in the art that modifications and variations may be made in the present disclosure without departing from the scope or spirit of the subject matter. For instance, features illustrated or described as part of one embodiment, may be used in another embodiment to yield a still further embodiment. Thus, it is intended that the present disclosure cover such modifications and variations as come within the scope of the appended claims and their equivalents.

The present disclosure is generally directed to PCR primers as may be utilized in detection of the genes encoding the thermostable direct hemolysin (tdh) and the TDH-related hemolysin (trh). Commonly used PCR primers for amplification of these genes for screening purposes have been prone to false negative reactions (i.e., no PCR product from samples in which pathogenic V. parahaemolyticus strains are present). According to the present disclosure, two new sets of PCR primers for the detection of the tdh and trh genes have been developed (SEQ ID NO: 1-SEQ ID NO: 4 as described in Table 1, below.

TABLE 1

| Primer | SEQ ID NO | Sequence |
|---|---|---|
| tdh86F | SEQ ID NO: 1 | CTGTCCCTTTTCCTGCCCCCG |
| tdh331R | SEQ ID NO: 2 | AGCCAGACACCGCTGCCATTG |
| trh90F | SEQ ID NO: 3 | ACCTTTTCCTTCTCCWGGKTCSG |
| trh500R | SEQ ID NO: 4 | CCGCTCTCATATGCYTCGACAKT |
| L-tdh | SEQ ID NO: 5 | GTAAAGGTCTCTGACTTTTGGAC |
| R-tdh | SEQ ID NO: 6 | TGGAATAGAACCTTCATCTTCACC |
| L-trh | SEQ ID NO: 7 | TTGGCTTCGATATTTTCAGTATCT |
| R-trh | SEQ ID NO: 8 | CATAACAAACATATGCCCATTTCCG |

Analysis using these new primers can provide improved detection, including fewer false negatives, as compared to previously known PCR primers for tdh and trh detection. For example, and as described further in the Example section, below, a screening process utilizing the disclosed PCR primers detected 23 tdh positive and 4 trh positive strains in a collection of 48 environmental V. parahaemolyticus isolates. The identities of all PCR products were confirmed by DNA sequence analysis. Screening the same set of isolates with previously described sets of primers (SEQ ID NO: 5-SEQ ID NO: 8 as described by Bej, A. K., D. P. Patterson, C. W. Brasher, M. C. L. Vickery, D. D. Jones, and C. A. Kaysner. 1999. J. Microbiol. Methods 36:215-225) yielded only 11 tdh positive and 2 trh positive strains. These new sets of PCR primers will facilitate the detection of potentially pathogenic strains of V. parahaemolyticus.

The PCR reaction conditions of the method of detection of pathogenic strains of V. parahaemolyticus generally encompass standard PCR reaction conditions of the various types of PCR (e.g. nested PCR, multiplex PCR, micro-PCR, single PCR) or partial variations thereof, and within the range of variations that can easily be envisaged by one skilled in the art.

A method for detection of a pathogenic strain of V. parahaemolyticus can include incubating a test sample under amplification conditions such that one or both of the disclosed primer pairs hybridize to denatured tdh and/or trh strands in the sample following which a polymerase included in the reaction mixture can extend the primers. Following, detection of the presence or quantity of amplified tdh and/or trh segments in the test sample can be carried out.

The disclosed primers can amplify relatively large segments of the tdh and trh genes with high sequence identity. For instance, the SEQ ID NO: 1 and SEQ ID NO: 2 PCR primer pair can amplify a 245 bp segment of the tdh gene and the SEQ ID NO: 3 and SEQ ID NO: 4 PCR primer pair can amplify a 410 bp segment of the trh gene with greater than about 70%, greater than about 80%, or greater than about 90% sequence identity.

The PCR amplification process can utilize any PCR amplification methodology as is generally known in the art including, for example, thermal block PCR or micro-PCR device, but is not limited thereto. For example, in the PCR amplification step, the reaction mixture can be repeatedly cycled between a low temperature, generally of from about 37° C. to about 70° C., for primer annealing to the selected target sequence or for strand reassociation, an intermediate temperature, generally of from about 70° C. to about 80° C., for polymerase extension of the primers, and a higher temperature, generally of from about 80° C. to about 100° C., for denaturation, i.e. separation of the strands. Although three temperature ranges have been described, it is often possible that the amplification process can be adequately conducted between two of the temperature ranges. Each thermal cycle of the two or three temperatures can increase the concentration of the amplified target DNA sequence as much as two-fold, so that every series of ten amplification cycles can increase the concentration as much as 1024-fold. If a thermostable DNA polymerase, such as that purified from the bacterium Thermus aquaticus (Taq) is used, the polymerase reaction can be cycled many times, for instance from about 20 to about 40 times, between the two or three temperatures without need to augment the initially added polymerase enzyme.

The sample size subjected to the amplification process can vary, as is known in the art. In general, the sample volume can be on the order of about 0.1 ml to about 1.0 ml, though smaller or larger test volumes can be utilized according to known practice. For instance, in one embodiment, a micro-PCR device can be utilized in carrying out the PCR amplification. A micro-PCR device uses a small chip for PCR and is able to formulate small test sample sizes, for instance about less than about 10 μl, less than about 5 μl, or about 1 μl can be injected into the device for analysis. The method has the advantages of using a small amount of reaction composition and monitoring ease. The chip can be mounted on a module and the DNA amplification can be monitored in real-time during the PCR reaction. GenSpector™ TMC-1000 (Samsung Advanced Institute of Technology, Korea) is an example of a commercially available micro-PCR device as may be utilized.

Depending upon the specific conditions of the detection process, a detection method can include steps in addition to the amplification process. For instance a method can include initial recovery of V. parahaemolyticus from a large test sample, for instance as may be obtained from an environmental testing site. The initial test sample can be obtained from any source in which V. parahaemolyticus may be discovered, for instance any source from which seafood may be farmed, harvested, or stored. A method can also include treatment of the initially obtained test sample so as to recover and concentrate trh and/or tdh in the test sample.

The operation of recovering V. parahaemolyticus from a test sample may be performed by any of several suitable means, including, for example, filtration and centrifugation, possibly with the help of suspended or dissolved additives which serve to capture or flocculate the organisms in a physical state which facilitates their separation. If the V. parahaemolyticus are not adsorbed to larger particles or flocculated, the nominal filter pore size can be on the order of about 0.2 to 0.5 μm, for instance about 0.45 μm, to assure efficient capture. If the V. parahaemolyticus are recovered in a gel or adsorbed to particles, much larger filter pore sizes can be used to accelerate filtration.

The captured V. parahaemolyticus can be treated in such a manner so as to recover undegraded tdh and/or trh sequences from the sample. This may be performed by any of many suitable methods. By way of example, recovery of the tdh and/or trh sequences of a test sample may be carried out by microbial lysis that may be effected by brief exposure to extremes of pH, organic solvents, chaotropic agents like urea and guanidine HCl, detergents like sodium dodecyl sulfate (SDS) and Triton X-100, osmotic shock, lysozyme digestion, or protease digestion and the like. Interfering substances can be removed, for example, by organic solvent extraction, acid precipitation, ultrafiltration, solid-phase extraction, HPLC, LiCl precipitation, protease digestion, RNase digestion, or polyethylene glycol precipitation and the like. Solid-phase extraction or HPLC can be based on ion-exchange, reverse-phase, hydrophobic-interaction, or silica-gel adsorption interactions.

In one embodiment, the test sample can be treated so as to recover tdh and/or trh from the bacteria of the sample, for instance by lysis, such that essentially all undegraded tdh and/or trh sequences are recovered so as to be sufficiently free of potentially interfering substances, such as enzymes, low molecular weight inhibitors or other components that might interfere with enzymatic amplification of the tdh and/or trh sequences. Following PCR amplification of the tdh and/or trh sequences, amplified DNA can be detected by detection methods as are generally known. For example, detection can be by means of suitable hybridization probes utilizing probes of specific sequences from the tdh and/or trh sequences. Quantification of the amplified target DNA sequences may also be carried out, if desired.

Separation of the amplified PCR product, side products, and unreacted reagents by HPLC can provide a rapid quantitative report on the presence or absence of the amplified DNA of the expected size range. HPLC columns may, for example, be based on ion exchange, paired-ion reverse-phase, or size exclusion separations. The column effluent is generally most simply detected and quantified by ultraviolet absorbance in the 250-280 nm spectral region, although fluorescent monitoring, after post-column derivatization with a fluorescent DNA-binding dye, and electrochemical detection also are possible and generally are potentially more sensitive than spectrophotometry. Separation of amplified PCR product, side products, and unreacted reagents by gel electrophoresis, followed by DNA staining with a fluorescent or absorbing dye, also can provide information with regard to the presence or absence of amplified tdh and/or trh sequences in the expected size range.

In one embodiment, detection can be carried out via hybridization of the amplified product to a single-stranded oligonucleotide probe. The probe can be sequence-complementary to a DNA subsequence that is located between sequences of the gene that are complementary to the two primers of the PCR pair. In those embodiments in which the PCR amplified target DNA sequence is denatured and captured on a solid support, such as a nylon or nitrocellulose membrane, the probe may be radioactively tagged or attached directly or indirectly to an enzyme molecule. Then, after incubation of membrane-captured PCR amplified target DNA sequence product with the probe under hybridization conditions, excess probe can be washed away and detection can be by autoradiography or radiation counting, radioactive probe. In one embodiment, the probe can be optically detectable, for instance the probe can be directly or indirectly bound to a chromogenic or fluorogenic substrate for optical detection. In those embodiments in which the oligonucleotide hybridization probe has been attached to a solid support, the incubation of denatured PCR amplified target DNA sequence product with the solid support under hybridizing conditions can result in immobilization of the PCR product. In those embodiments in which the PCR product contains biotin or some other chemical group for which there are specific binding molecules, like avidin or antibodies, the immobilized product can be detected with an enzyme attached to the specific binding molecule, such as horseradish peroxidase or alkaline phosphatase attached to streptavidin.

Also described herein are kits for detection of pathogenic V. parahaemolyticus that include one or more of SEQ ID NO: 1, SEQ ID NO: 2, SEQ ID NO: 3, and SEQ ID NO: 4 for use in detecting tdh and/or trh in a sample. Use of the disclosed primers can provide testing kits with improved sensitivity and fewer false negative reactions. A detection kit can include one or both of the disclosed PCR primer pairs for detecting V. parahaemolyticus. A detection kit can also include the usual components (reacting buffer, Taq DNA polymerase, labeling material, control sequence for use as a probe, etc.) of a PCR microbial detection kit.

The test kits may comprise published instructions and reagents for the PCR amplification and detection of the targeted DNA sequence. In addition to the aforementioned primer pairs and probe sequence, the test kit may also include other reagents for the PCR amplification of the targeted DNA sequence, such as for example, lysing agents, PCR amplification polymerase and the like, and filtration devices for water sample collection.

The present disclosure may be better understood by reference to the Example, set forth below.

### Example

New primers for PCR amplification of tdh and trh genes were employed to screen environmental Vibrio parahaemolyticus strains. 23 tdh and 4 trh positive strains were detected from a collection of 48 strains. Previously described primers yielded 11 tdh and 2 trh positive results. The new primers were shown to facilitate detection of pathogenic V. parahaemolyticus.

A total of 48 environmental strains of V. parahaemolyticus that were isolated from a pristine estuary were screened via PCR using both the commonly used Bej et al. (1999) tdh and trh primers (SEQ ID NO: 5, SEQ ID NO: 6, SEQ ID NO: 7, SEQ ID NO: 8) and two newly designed primer sets (SEQ ID NO: 1, SEQ ID NO: 2, SEQ ID NO: 3, SEQ ID NO: 4). The new tdh and trh primers were found to be substantially more effective in the detection of these genes from environmental strains of V. parahaemolyticus.

All environmental V. parahaemolyticus strains were isolated from the Crab Haul Creek drainage in the North Inlet estuary, near Georgetown, S.C. (33° 20′ N, 79° 12′ W). This is a euhaline, highly productive saltmarsh ecosystem having semi-diumal tides (Buzzelli et al., 2004). Samples of intertidal creek water, bulk sediment, and sediment lining the burrows of fiddler crabs (Uca pugilator and Uca pugnax) were collected in August and September, 2011. Samples were serially diluted with l×phosphate buffered saline (400 mM NaCl, 1.75 mM NaPO₄, pH 7.4). V. parahaemolyticus and related Vibrio strains were isolated by spread plating (without enrichment) onto Thiosulfate Citrate Bile Salts Sucrose (TCBS) (BD, NJ). TCBS agar plates were incubated at 35-37° C. for 48 h, according to the FDA protocol (Kaysner and DePaola, 2004), and green colonies of approximately 2-3 mm were sampled and streaked for purity on TCBS plates. An additional 11 strains, previously isolated from roots of the salt marsh plants Spartina alterniflora (smooth cordgrass) and Juncus roemerianus (black needlerush) (Bagwell et al., 1998; Gamble and Lovell, 2011) and one strain isolated from sediment in the growth zone of Juncus roemerianus in 2010 were also included in this analysis. V. parahaemolyticus ATCC 33846 was the positive control strain for tdh amplifications and V. parahaemolyticus ATCC 17802T was the positive control for trh amplifications.

Primers were designed utilizing tdh and trh sequences available in the NIH Genbank as of June, 2010. Sequences were aligned using Mega version 4.0 (Tamura et al., 2007) and priming sites were selected based on conserved regions to facilitate amplification of the most divergent sequences possible while still yielding a relatively large and informative portion of each gene.

The tdh primers tdh86F (SEQ ID NO: 1) and tdh331R (SEQ ID NO: 2) (Table 1) are 21 nucleotides in length and numbered based upon the V. parahaemolyticus ATCC 33846 tdh sequence (accession number GU971653). The trh primers trh90F (SEQ ID NO: 3) and trh500R (SEQ ID NO: 4) (Table 1) are 23 nucleotides in length and numbered based upon the V. parahaemolyticus ATCC 17802 trh sequence (accession number GU971654). Oligonucleotide primers were synthesized by Eurofins MWG Operon (Huntsville, Ala.).

All V. parahaemolyticus strains were grown in saline Luria Broth (per L: 10 g tryptone, 5 g yeast extract, 27 g NaCl) overnight at 37° C. with shaking. DNA was extracted using the Wizard Genomic DNA purification kit (Promega, Madison, Wis.) with minor modifications.

PCR reactions of 25 μl contained 1×PCR buffer (Valencia, Calif.), 1.25 units of Taq DNA polymerase (Qiagen), 0.5 μM of each primer (tdh86F (SEQ ID NO: 1) and tdh331R (SEQ ID NO: 2) or trh90F (SEQ ID NO: 3) and trh500R (SEQ ID NO: 4), 200 μM of each dNTP (premixed, Qiagen). The reaction conditions used to amplify tdh and trh employing the new primers were an initial denaturation at 95° C. for 5 min, followed by 40 cycles consisting of 95° C. for 1 min., 62° C. for 1 min, 72° C. for 1 min, and a final elongation of 72° C. for 2 min. PCR reactions separately targeting tdh and trh were carried out using L-tdh (SEQ ID NO: 5), R-tdh (SEQ ID NO: 6), L-trh (SEQ ID NO: 7) and R-trh (SEQ ID NO: 8) for the conditions described by Bej et al. (1999). The products of the L-tdh (SEQ ID NO: 5) and R-tdh (SEQ ID NO: 6) corresponding to the expected size were recovered from (1%) agarose gels using the Wizard Gel Excision and PCR Clean Up Kit (Promega).

Primers tdh86F (SEQ ID NO: 1) and tdh331R (SEQ ID NO: 2) were employed to amplify a 245 base pair segment of the tdh gene which could be cleanly resolved as a single band on a 1% agarose gel (FIG. 1). Sequences were amplified from 23 of 48 strains (48%). In contrast, using the L-tdh (SEQ ID NO: 5)/R-tdh (SEQ ID NO: 6) primers only 11 of the isolates (23%) yielded a product that could be resolved. Both tdh primer sets amplified the tdh gene from the positive control strain ATCC 33846. However, attempts to amplify tdh from environmental strains with L-tdh (SEQ ID NO: 5)/R-tdh (SEQ ID NO: 6) resulted in the formation of spurious products (FIG. 1). This necessitated band purification and re-amplification before sequence data could be obtained to confirm the identity of the amplicon. All 23 tdh sequences when translated produced an 80 amino acid segment of the TDH protein that contained the conserved residues Arg46, Gly62 and Trp65 (Yanagihara et al., 2010). All of these tdh sequences had at least 92% sequence identity to the ATCC 33846 tdh with the exception of sequence JPW-9-11-9 which had 70% sequence identity.

Oligonucleotide primers trh90F (SEQ ID NO: 3) and trh500R (SEQ ID NO: 4) amplified a 410 bp segment of the trh gene. All sequenced TRH gene segments from North Inlet strains contained the conserved amino acid residues W65 and L66 (Kishishita et al., 1992) and trh gene sequences shared at least 92% sequence identity to the positive control (ATCC 17802). The new primers successfully amplified trh from 4 of 48 strains screened (8.3%) while L-trh (SEQ ID NO: 7) and R-trh (SEQ ID NO: 8) amplified the trh gene from only 2 of the strains (4.1%). Both sets of trh primers yielded single bands for all trh gene sequences that were amplified (FIG. 1).

The new sets of primers described here proved more effective in detecting the presence of the tdh and trh genes from environmental isolates than primers described previously. This should facilitate a more accurate assessment of the presence of potentially pathogenic strains of V. parahaemolyticus and could aid in the identification of emerging pathogenic strains.

Those skilled in the art will readily appreciate that many modifications are possible in the exemplary embodiments that have been described in detail above without materially departing from the novel teachings and advantages of this disclosure. Accordingly, all such modifications are intended to be included within the scope of this invention which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present invention.

What is claimed is:

1. A PCR primer for detecting a pathogenic strain of V. parahaemolyticus consisting of SEQ ID NO: 1.

2. A PCR primer for detecting a pathogenic strain of V. parahaemolyticus consisting of SEQ ID NO: 2.

3. A PCR primer for detecting a pathogenic strain of V. parahaemolyticus consisting of SEQ ID NO: 3.

4. A PCR primer for detecting a pathogenic strain of V. parahaemolyticus consisting of SEQ ID NO: 4.

5. A kit for detecting a pathogenic strain of V. parahaemolyticus, the kit comprising a pair of PCR primers, the pair of PCR primers consisting of SEQ ID NO: 1 and SEQ ID NO: 2 or SEQ ID NO: 3 and SEQ ID NO: 4.

6. The kit according to claim 5, the kit comprising a first and second pair of PCR primers, the first pair of PCR primers consisting of SEQ ID NO: 1 and SEQ ID NO: 2, and the second pair of PCR primers consisting of SEQ ID NO: 3 and SEQ ID NO: 4.

7. The kit according to claim 5, further comprising Taq DNA polymerase.

8. The kit according to claim 5, further comprising a probe for a tdh or a trh sequence.

9. The kit according to claim 8, further comprising a chromogenic or fluorogenic substrate for optical detection of the probe.

10. A method for detecting a pathogenic strain of V. parahaemolyticus, the method comprising amplifying a tdh sequence contained in a test sample by use of a pair of PCR primers consisting of SEQ ID NO: 1 and SEQ ID NO: 2.

11. A method for detecting a pathogenic strain of V. parahaemolyticus, the method comprising amplifying a trh sequence contained in a test sample by use of a pair of PCR primers consisting of SEQ ID NO: 3 and SEQ ID NO: 4.
